(19) United States
(12) Patent Application Publication
(10) Pub. No.: US 2021/0338128 A1
(43) Pub. Date: Nov. 4, 2021

(54) Sensor for Measuring a Biological Potential

(71) Applicant: Urgotech, Paris (FR)

(72) Inventors: Guirec Le Lous, Paris (FR); Robin Reynaud, Asnieres Sur Seine (FR)

(73) Assignee: Urgotech, Paris (FR)

(21) Appl. No.: 17/279,560

(22) PCT Filed: Oct. 2, 2019
(86) PCT No.: PCT/FR2019/052324

§ 371 Date: Mar. 24, 2021

(30) Foreign Application Priority Data
Oct. 2, 2018 (FR) 18 59133

(51) Int. Cl. A61B 5/256; A61B 5/00; A61B 5/16; A61B 5/282; A61B 5/291

(52) U.S. Cl. CPC A61B 5/256; A61B 5/4088; A61B 5/4806; A61B 5/168; A61B 2562/227; A61B 5/291; A61B 5/6803; A61B 2560/04; A61B 2562/125; A61B 5/282

(57) ABSTRACT

A sensor (30) for measuring a biological potential of an individual, comprising: a measurement electrode (31) comprising a base (32), at least one leg intended to come into contact with an anatomical zone of the individual and at least one electrical connection member (38), at least one locking member (45) configured to cooperate in a reversible manner with at least one complementary locking member (25) on an external support (10) so as to removably fasten the sensor (30) to the external support (10), the locking member (45) being separate from the electrical connection member (38), a protection element (40) made from a resilient material and delimiting a recess (41) in which the leg extends.

### TECHNICAL FIELD p The present invention relates to a sensor for measuring a biological potential, such as the production of an electroencephalogram or an electrocardiogram, in humans or animals.

### PRIOR ART

The control, the characterization, the monitoring, even the feedback of health parameters such as cardiac or cerebral parameters at present forms the basis of the main preoccupations of the medical device industry.

One of the current lines of thought in these new approaches is to be able to characterize the sleep of an individual. To this end, it is known for sleep spindles to be identified. Sleep spindles are signals of electrical brain activity with frequencies that generally range between 9 and 16 Hz (Molle et al, 2011) and with an amplitude ranging from 25 to 150 microvolts. Low frequency and high frequency sleep spindles are found, which are variable and specific to each individual. Sleep spindles generally last between 0.5 to 2 seconds and are the product of activity of the reticulo-thalamo cortical network. It has been shown that the production of sleep spindles with a high density is associated with effective sleep.

It is possible to identify sleep spindles by acquiring brain activity signals at specific positions of the head of the user, in particular positions C3, C4 and/or Cz defined by the “international 10/20 system” (see document WO-A1-2009/061920 in particular).

The international 10/20 system is particularly mentioned through patent application WO-A1-2009/061920. This system, illustrated in FIG. 1 (according to the modified combinatorial nomenclature, Directives N° 5: Guidelines for Standard Electrode Position Nomenclature, American Clinical Neurophysiology Society, 2006), ultimately is an internationally recognized method for locating the possible position of at least one electrode on the surface of a human skull within the context of producing an electroencephalogram. In this system, each measurement electrode is thus identified by a letter coding its position in relation to the large brain regions, and a number or the letter z that defines the hemisphere (Jasper, 1958):
the letters F, T, C, P and O respectively indicate the Frontal, Temporal, Central, Parietal and Occipital regions;
the even numbers (2, 4, 6, 8) correspond to the right-hand hemisphere;
the odd numbers (1, 3, 5, 7) correspond to the left-hand hemisphere;
the letter z indicates the electrodes located on the median line.

Another potential application relates to attention deficit disorders with or without hyperactivity (ADHD). In this application, the acquisition of brain activity signals also can occur at positions C3, C4, and/or Cz (optionally and/or CPz and/or FCz) of the international 10/20 system.

Yet another application for its part relates to memory disorders, in particular within the context of Alzheimer's disease. In this application, the acquisition of brain activity signals can, for its part, particularly occur at positions C3, C4, T7 and/or T8 of the international 10/20 system, with positions T7 and T8 respectively corresponding to positions T3 and T4 according to a previous simplified nomenclature of this international 10/20 system. A seriously considered variant could involve acquiring these brain activity signals at positions C3, C4, CPz and/or FT7 of the international 10/20 system.

Conventionally, the measurement electrodes used in these applications can be wet or dry. Wet electrodes have the disadvantage of soiling the skull and the scalp where they are directly attached, despite a highly perceptible reception quality of the desired signal. Dry electrodes, for their part, do not exhibit this discomfort. However, their hardness can be a disadvantage in the event of excessive contact pressure being exerted by the helmet supporting said electrode, thus making wearing the helmet unpleasant for the user. Therefore, movable or conformable electrodes made of metal or conductive polymer have been developed in order to attempt to overcome this disadvantage.

A particularly symbolic example of this desire to produce dry conformable electrodes without causing any discomfort for the user is described in patent application EP 2 827 770 by Cognionics. To this end, the inventors have implemented tabs made of elastomer material that experience an external or lateral movement (S) perpendicular to the exerted pressure (P).

According to another variant published in literature, application WO 2009/134763 by the University of Rhode Island proposes an electrode, the metal tabs of which are rendered compressible by means of a spring inserted directly inside their structure, causing them to move in a direction parallel to the exerted pressure constraint.

However, in both cases these electrodes present a limit to this comfort solution, particularly when the tab is at the maximum of its lateral movement or when the movable part of the tab fully enters into the immovable part thereof, then respectively leaving either the base or the immovable part of the electrode directly in contact with the scalp.

In order to overcome this disadvantage, some manufacturers have proposed introducing a protective element having a cavity integrating the measurement electrode, which cavity itself has borders presenting a lateral thickening and acting as a peripheral damper for the electrode. To this end, application WO 2012/156499 describes the introduction of such a means. An additional major advantage that is also provided by this protective element is that it allows the number of measurement artefacts to be drastically reduced due to an improvement in the stability of the electrode on the skull of the user.

However, this solution is not perfect, the measurement electrode still can have a certain degree of mobility on the external support on which it is attached, thus leaving the possibility of the electrode disconnecting from this support.

Therefore, the subject matter of the present invention is to propose a sensor for measuring a biological potential, said sensor advantageously having improved comfort qualities for the user and an assured engagement, reducing the measurement artefacts of the desired biological potential.

### PRESENTATION OF THE INVENTION

Thus, according to a first aspect, the subject matter of the invention involves a sensor for measuring a biological potential of an individual, the sensor being intended to be placed on an anatomical zone of the individual by means of an external support, the sensor comprising:
a measurement electrode adapted to conduct an electric potential, said measurement electrode comprising:
a base;
at least one tab extending from the base to a free contact part intended to come into contact with the anatomical zone of the individual, the tab being configured so that at least the contact part can move relative to the base; and
at least one electrical connection member configured to cooperate with at least one complementary electrical connection member on the external support;
at least one locking member configured to reversibly cooperate with at least one complementary locking member on the external support so as to detachably fix said sensor to the external support, the locking member being separate from the electrical connection member;
said sensor further comprising a protective element extending from the base and defining a cavity, into which the tab extends, the protective element being configured to be flush with the contact part of the tab when the sensor is placed on the anatomical zone, the protective element being produced from a resilient material.

The Applicant has highlighted the advantage of developing a locking member separate from the electrical connection member of the measurement electrode for reliably and reproducibly fixing the sensor to any external support, such as a helmet, a bracelet or a belt, and to thus reduce the measurement artefacts of the biological potential.

The protective element fulfils a role of damper and of peripheral stabilizer for the sensor and, more specifically, for the measurement electrode. This is more specifically verified for the measurement electrodes, the base of which would come into immediate contact with the surface of the user, for which electrodes the one or more tabs move laterally once the pressure is exerted or the electrodes for which the immovable attachment part would exert this contact, for which electrodes the movable contact part fully enters its constituting immovable attachment part once the pressure is exerted.

This role of damper and of stabilizer appears to be particularly advantageous for at least two reasons:

in terms of maintaining the comfort of the sensor on the contact surface of the user, with the electrode no longer having a painful projecting point beyond this damping part;

in terms of measurement, with the sensor being securely attached to the external element that supports it, the measurement artefacts will be reduced in terms of their number.

The sensor can have a longitudinal axis and the locking member and the complementary locking member can be configured to form a bayonet attachment. The locking member comprises at least one locking surface extending transversely in relation to the longitudinal axis and facing the base in a direction opposite to the tab, the locking surface being configured to cooperate with a locking edge as a complementary locking member on the external support, the locking edge defining a locking opening, the locking member and the locking edge being shaped to allow the locking surface to pass through the locking opening in a first angular position of the locking member in relation to the locking edge along the longitudinal axis, and to prevent the locking surface from passing through the locking opening in a second angular position of the locking member in relation to the locking edge along the longitudinal axis, the locking surface being in abutment on the locking edge when the sensor is fixed onto the external support in the second angular position.

The locking member can comprise at least two locking rods extending from the base along the longitudinal axis, with each locking rod having the locking surface.

The locking member can comprise an annular locking skirt around the longitudinal axis and from which at least two lugs radially extend so as to each have the locking surface.

As an alternative embodiment, the sensor can have a longitudinal axis and the locking member can comprise at least one locking rod extending from the base along the longitudinal axis in a direction opposite to the tab and a detachable pin. The locking rod comprises a locking orifice extending transversely in relation to the longitudinal axis, the locking rod and the pin being configured to cooperate with a locking hole as a complementary locking member on the external support, the pin extending into the correspondingly placed locking orifice and locking hole when the sensor is fixed onto the external support.

According to another alternative embodiment, the sensor can have a longitudinal axis and the locking member can comprise a thread on a lateral wall extending from the base along the longitudinal axis in a direction opposite to the tab. The thread is adapted to cooperate with a complementary thread as a complementary locking member on the external support.

According to another alternative embodiment, the sensor can have a longitudinal axis and the locking member can be a clip. The clip comprises at least one locking rod extending from a surface of the base opposite to the tab, with the locking rod being configured to cooperate with a locking edge as a complementary locking member on the external support, the locking rod having a rest position, in which said locking rod extends along the longitudinal axis and has a locking surface transverse to the longitudinal axis and facing the base, the locking rod being resiliently deformable in order to be spaced apart from the rest position, the locking surface being in abutment on the locking edge when the sensor is fixed onto the external support.

According to another alternative embodiment, the sensor can have a longitudinal axis and the locking member can comprise a crimping skirt extending from a surface of the base opposite to the tab. The crimping skirt is configured to cooperate with a locking edge as a complementary locking member on the external support, the crimping skirt having an assembly state, in which said crimping skirt defines a housing around the longitudinal axis that is adapted to receive the locking edge, the crimping skirt being deformable so as to have at least one locking surface transverse to the longitudinal axis and being arranged to retain the crimping edge in the housing when the sensor is fixed onto the external support.

In one embodiment, the base can have a central axis and the tab can comprise an attachment part secured to the base, the contact part being mounted so as to translationally move along the central axis on the attachment part.

The tab then can comprise a resilient element inserted between the attachment part and the contact part.

In another embodiment, the base can have a central axis and the contact part can be translationally movable in relation to the central axis.

In one embodiment, the measurement electrode can be a wet or dry electrode, preferably said measurement electrode will be a dry electrode.

A “wet electrode” is understood to be any electrode necessarily implementing a conductive gel or paste, for example, based on electrolytes, at the interface of a contact surface to which it is attached.

A “dry electrode” is understood to be any electrode based on a material that is conductive or that is rendered conductive, selected from metal, metal alloys, elastomers or plastics, having a certain hardness and for which the use of a conductive gel or paste is not necessary.

The protective element then can comprise the locking member. According to this arrangement, the locking member forms an integral part of the protective element, thus allowing optimal comfort and measurement stabilization of a biological potential of interest.

The resilient material can have a Young's modulus ranging between 10 Kpa and 100 MPa, in particular between 10 Kpa and 80 MPa, with the resilient material particularly being a silicone, a plastic material or an elastomer.

A “resilient material” is understood to be any compressible or deformable material with the property of returning to its initial volume and/or its initial shape once the external physical constraint is removed. In other words, a resilient material is understood to be a shape memory material.

The relevant polymers with respect to the production of the protective element can include the elastomers that are defined as being thermoplastics, the polymers that are defined as being resins or even the silicones as described hereafter.

In a particularly advantageous manner, the measurement electrode comprises at least three tabs, more preferably at least six tabs.

Advantageously, the measurement electrode is produced from a conductive material or a material that is rendered conductive and is selected from the list of materials formed by at least one metal material or a metal alloy and/or at least one polymer.

Among the metal materials that are used within the scope of the present invention, materials such as steel, preferably stainless steel, tin, copper, silver, platinum, titanium, lead, gold, zinc, aluminum, iron, chrome, the derivatives thereof or the alloys thereof are particularly favored.

The relevant polymers with respect to the production of the measurement electrode within the meaning of the present invention can include the elastomers defined as thermoplastics, the polymers defined as being resins, or even silicones.

The thermoplastic elastomers can include olefin, styrene, ester, polyamide, polyurethane or even polyvinyl based elastomers.

A non-limiting example of resin can include the following: acrylonitrile-styrene (AS), acrylonitrile butadiene (ABS) based resins, epoxy resins, tetrafluoroethylene and ethylene (ETFE) based resins, those based on tetrafluoroethylene, and hexafluoropropylene (FEP), those based on hexafluoropropylene and ethylene (EFEP), those based on polyvinylidene fluoride (PVDF), poly chl orotrifluoro ethylene (PCTFE), chlorotrifluoroethylene and ethylene (ECTFE), polycaproamide (nylon 6), polyhexamethylene adipamide (nylon 66), polytetramethylene adipamide (nylon 46), polyhexamethylene men len sebacamide (nylon 610), polyhexamethylene men len dodecamide (nylon 612), polydodecane amide (nylon 12), polyundecane amide (nylon 11), terephthalamide, poly-xylylene adipamide (nylon XD6), polynonamethylene terephthalamide, polyundecanamide terephthalamide (nylon 11T), polydecamethylene decanamide (nylon 1010), polydecamethylene dodecanamide (nylon 1012) and elastomer based on amide (TPA), polybutylene terephthalate (PBT), polybutylene naphthalate (PBN), polyethylene naphthalate (PEN), polycarbonate (PC), linear low density polyethylene (LLDPE), very low density polyethylene, or even low density polyethylene (LDPE), medium density polyethylene (MDPE), high density polyethylene (HDPE), based on vinyl acetate copolymer (EVA), vinyl alcohol (EVOH or BVOH), polyvinyl alcohol (PVA), polybutene (PB), polymethylpentene (PMP), polyether ether ketone (PEEK), polyether sulfone (PES), polyethylene terephthalate (PET), polyimide (PI), polyetherimide (PEI), acrylic resin (PMMA), polyacetal (POM), polypropylene (PP), polyphenylene sulfide (PPS), polystyrene (PS), polysulfone (PSU), polytetrafluoroethylene (PTFE), poloxamer, the derivatives of cellulose such as hydroxypropylcellulose (HPC), or hydroxymethylcellulose (HMC), polypropyleneglycol (PPG), polyethylene glycol (PEG) or polyvinyl chloride (PVC).

Among the silicones that can be used within the scope of the present invention, silicones based on siloxanes or polysiloxanes and their derivatives, such as, for example, polydimethylsiloxane, are preferably used.

According to a particular embodiment of the invention, the relevant polymers implemented in the production of the measurement electrode exhibit hardness that is within a range ranging from 10 Shore A to 80 Shore A.

The hardness of said polymers particularly can be less than or equal to 65 Shore A. When the material forming the measurement electrode is rendered conductive, said material comprises conducting particles such as graphite, electrolytes, particles of metal or of metal alloy, active carbon, organic conducting powders, or carbon, optionally in the form of nanotubes.

Alternatively or additionally, the material forming the electrode can be rendered conductive by the at least partial application of a conducting layer, particularly selected from a paint, an ink, a glue or a conductive adhesive. In particular, the conducting layer can comprise silver, silver salts, derivatives of silver or a silver alloy. The conducting layer also can comprise a conductive polymer, preferably a mixture of poly(3,4-ethylenedioxythiophene) (PEDOT) and of sodium poly(styrene sulfonate) (PSS). By way of a non-limiting example, a silver chloride based ink is particularly preferred.

In one embodiment, the electrical connection member of the measurement electrode is formed by a part made of conductive material overmolded by the material forming the measurement electrode or is produced by molding directly from the material forming the measurement electrode.

The electrical connection member is intended to at least partially provide a conductive type contact with the external support. On its own it is not intended to act as a locking member with this external support.

According to an alternative embodiment of the invention, when the electrical connection member is produced from an overmolded part, said part is manufactured from at least one metal material or from at least one metal alloy.

Among the metal materials used within the scope of the production of the electrical connection member, materials such as steel, preferably stainless steel, tin, copper, silver, platinum, titanium, lead, gold, zinc, aluminum, iron, chrome, the derivatives thereof or the alloys thereof are particularly favored.

According to a preferred embodiment of the invention, the electrical connection member emerges from the base of the measurement electrode on the side opposite that from which the at least one tab extends.

The sensor can further comprise a casing accommodating the base, a portion of the tab and at least one portion of the electrical connection member of the measurement electrode, the casing having a dorsal surface intended to be placed facing the external support, and a frontal surface opposite to the dorsal surface, with the tab of the measurement electrode projecting in relation to the frontal surface of the casing, the protective element being secured to the casing and extending from the frontal surface of the casing.

The casing can comprise the locking member.

The locking member can extend from the dorsal surface of the casing.

The casing can comprise a frontal casing portion and a dorsal casing portion configured to be detachably assembled together by defining a housing accommodating the base, a portion of the tab and at least one portion of the electrical connection member of the measurement electrode, the frontal casing portion supporting at least one portion of the frontal surface of the casing and the dorsal casing portion supporting at least one portion of the dorsal surface of the casing, the frontal casing portion being provided with at least one orifice, through which the tab of the measurement electrode extends.

The dorsal casing portion can have a peripheral portion around a casing axis and a central portion centered on the casing axis, with the central portion being offset along the casing axis in relation to the peripheral portion in order to form a portion of the housing, the frontal casing portion being mounted on the central portion of the dorsal casing portion.

The protective element can comprise a bottom extending transversely in relation to a protective element axis and a lateral wall extending from the bottom around the protective element axis, with the protective element covering the frontal surface of the casing and being provided with at least one orifice arranged in the bottom and through which the tab of the measurement electrode passes.

According to a second aspect, the invention proposes a measurement assembly comprising a sensor as previously defined and an external support configured to be placed on an anatomical zone of the individual, the external support comprising:
at least one complementary electrical connection member configured to cooperate with the connection member of the sensor;
at least one complementary locking member configured to reversibly cooperate with the locking member of the sensor.

The external support can be selected from a helmet, a bracelet and a belt.

According to a third aspect, the invention proposes a measurement system comprising a measurement assembly as previously defined and a processing unit configured to receive the biological potential measured by the sensor.

### DESCRIPTION OF THE FIGURES

Further aims and advantages of the invention will become apparent from reading the following description of particular embodiments of the invention, which are provided by way of a non-limiting example, with the description being provided with reference to the accompanying drawings, in which:

FIG. 1 shows a general view of a system for measuring a biological potential, the measurement system comprising a sensor according to a first embodiment of the invention disposed on an external support, namely a helmet;

FIG. 2 shows a partial exploded view of the measurement system of FIG. 1, illustrating a bayonet locking member intended to cooperate with complementary locking openings on the helmet in order to fix the sensor onto the helmet;

FIG. 3 shows a section view, along the orientation referenced in FIG. 2, of the sensor;

FIG. 4 shows an exploded perspective view of a front face of a sensor according to a second embodiment of the invention;

FIG. 5 shows a perspective view of a rear face of the sensor of FIG. 4.

### DETAILED DESCRIPTION OF THE INVENTION

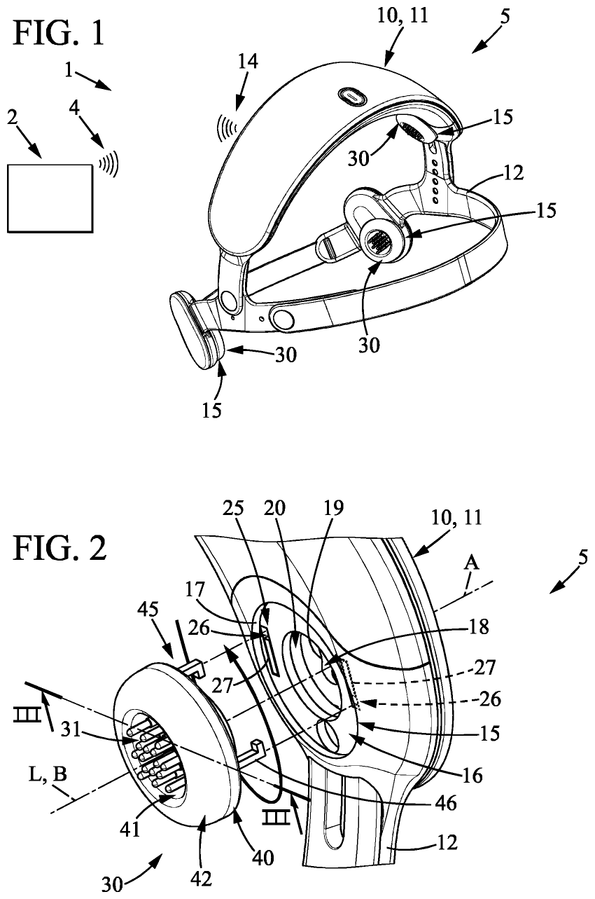

FIG. 1 shows a measurement system 1 for measuring a biological potential of an individual. Without being limited thereto, in the embodiment shown, the measurement system 1 is intended to allow the sleep of an individual to be characterized and, if applicable, to allow the individual to improve the quality of their sleep on the basis of the measured biological potential.

The measurement system 1 comprises a measurement assembly 5 configured to measure the biological potential representing brain waves of the individual and a processing unit 2 configured to use the biological potential measured by the measurement assembly 5.

In particular, the processing unit 2 can be adapted to identify the brain waves characterizing falling asleep and sleep, in particular the sleep spindles. The processing unit 2 also can be adapted to help the individual to promote the transmission of these brain waves.

The measurement assembly 5 comprises an external support 10, configured to be placed on an anatomical zone of the individual where the biological potential must be measured, and one or more sensors 30 detachably fixed onto the external support 10.

In the embodiment shown, in order to measure the brain waves, the external support is in the form of a helmet 11 comprising a frame 12, which preferably is adjustable, shaped so as to adapt to the skull of the individual. The helmet 11 has one or more sites 15 each configured to provide an electrical connection and a mechanical connection with one of the sensors 30.

In particular, sleep spindles can be identified by acquiring brain waves at specific positions of the skull of the individual, in particular at positions C3, C4 and/or Cz defined by the “international 10/20 system” (see, in particular, document WO-A1-2009/061920). In FIG. 1, the helmet 11 then comprises four sites 15 and four sensors 30: a site 15 and a sensor 30 in position C3, a site 15 and a sensor 30 in position C4, a site 15 and a sensor 30 in position Cz (not shown and not indicated on the present figure), and two sites 15 and two sensors 30 in two distinct positions located behind each ear of the individual and acting as a ground and/or reference. The number and the arrangement of the sites 15 could be different as a function of the contemplated application and of the biological potential to be measured.

In order to ensure the transmission of the data measured by the measurement assembly 5 to the processing unit 2, the helmet 11 can comprise a communication interface 14 configured to communicate with a communication interface 4 of the processing unit 2. In the embodiment provided, the communication interfaces 4, 14 provide a wireless communication. As an alternative embodiment, this communication could be wired.

In FIG. 2, each site 15 is formed by a hollow indentation 16 formed in an internal surface of the frame 12 intended to be placed facing the skull of the individual. The indentation 16 is defined by a cylindrical lateral surface 17 along an assembly axis A.

In order to provide the electrical connection at each of the sites 15, the helmet 11 comprises a complementary electrical connection member 18 configured to cooperate with a connection member 38 of the sensor 30. For example, the complementary electrical connection member 18 is in the form of a contact strip 19 made of conductive material centrally extending into a connection opening 20 provided in the indentation 16.

Furthermore, in order to provide the electrical connection at each of the sites 15, the helmet 11 comprises a complementary locking member 25 configured to reversibly cooperate with a locking member 45 of the sensor 30. For example, in the embodiment shown, as will become apparent from the remainder of the description, the locking member 45 and the complementary locking member 25 are shaped so as to form a bayonet attachment. The complementary locking member 25 of the helmet 11 then comprises two locking openings 26 provided in the indentation 16 on either side of the connection opening 20. Each locking opening 26 is defined by a locking edge 27.

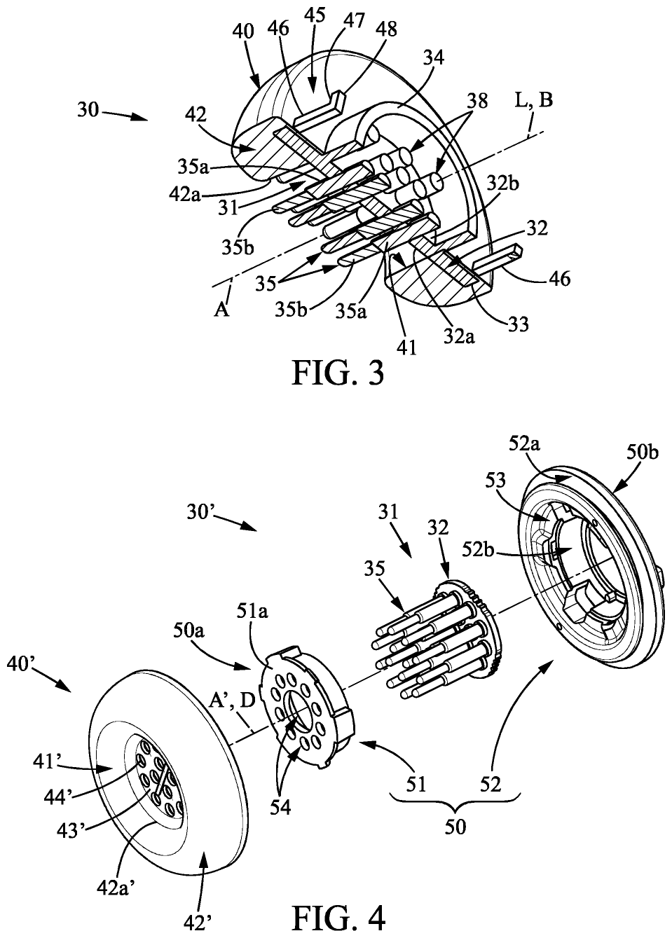

In FIGS. 2 and 3, each sensor 30 has a longitudinal axis L and is configured to be mounted on one of the sites 15 coaxial to the indentation 16.

The sensor 30 comprises a measurement electrode 31 adapted to conduct an electric potential. Preferably, the measurement electrode 31 is a “dry” electrode, i.e. produced from a material that is conductive or that is rendered conductive and that is selected from metal, metal alloys, elastomers or plastics, having a certain degree of hardness and for which the use of a conductive gel or paste is not necessary. In particular, when the material forming the measurement electrode 31 is rendered conductive, said electrode can comprise conducting particles such as graphite, electrolytes, particles of metal or of metal alloy, active carbon, conducting organic powders, or carbon, optionally in the form of nanotubes. Alternatively or additionally, the material forming the measurement electrode 31 can be rendered conductive by the at least partial application of a paint, an ink, a glue or even a conductive adhesive. By way of a non-limiting example, a silver chloride based ink is particularly preferred.

The measurement electrode 31 comprises a base 32 in the form of a plate extending transversally in relation to a central axis B coincident with the longitudinal axis L of the sensor 30. The base 32 has first 32a and second 32b surfaces that are opposite and are connected together by an external edge 33. In the embodiment shown, the first 32a and second 32b surfaces are flat and the external edge 33 defines a circular profile corresponding to the lateral surface 17 of the indentation 16. On the second surface 32b, a cylindrical fitting wall 34 along the central axis B is provided to cooperate with the connection opening 20 of the site 15 of the helmet 11.

The measurement electrode 31 comprises one or more tabs 35, preferably at least three and 16 in the embodiment shown, which extend from the first surface 32a of the base 32. In the embodiment shown, each tab 35 extends parallel to the central axis B of the base 32 and comprises an attachment part 35a secured to the base 32 and a contact part 35b mounted so as to translationally move along the central axis B on the attachment part 35a. The contact part 35b, which is free and movable in relation to the base 32, is intended to come into contact with the anatomical zone of the individual where the biological potential is measured. A resilient element, such as a helical spring, can be inserted between the attachment part 35a and the contact part 35b of the tab 35 in order to:

provide a resilient stress on the contact part 35b toward a deployed position, in which it is separated from the base 32 in the absence of an external constraint; and

allow the contact part 35b to move to a retracted position, in which it is brought closer to the base 32 under the effect of a pressure exerted by the anatomical zone when the helmet is placed on the skull of the individual.

In other embodiments, each tab 35 could be produced in any appropriate manner in order for the free contact part 35b to be able to move in relation to the base 32, parallel to the central axis B or transversally in relation to the central axis B.

In the embodiment shown, the electrical connection member 38 of the sensor 30 extends the attachment part 35a of each tab 35 through the base 32, so as to project in relation to the second surface 32b of the base 32. The tabs 35 are then arranged so that the electrical connection member 38 extends inside the fitting wall 34 of the base 32.

In one embodiment, the sensor 30 also comprises a protective element 40 extending from the base 32 and defining a cavity 41, in which the tabs extend 35. The protective element 40 provides a role of damper and of peripheral stabilizer for the sensor 30, and more specifically for the measurement electrode 31. The protective element 40 is produced from a resilient material, in particular a silicone, a plastic material or an elastomer. The resilient material can have a Young's modulus ranging between 10 KPa and 100 MPA, in particular between 10 KPa and 80 MPa.

In a first embodiment shown in FIG. 3, the protective element 40 is in the form of an annular bead 42 around a protective axis A, overmolded on a rim of the base 32 extending between the external edge 33 and the fitting wall 34. The bead 42 of the protective element 40 has a lateral wall 42a extending over the first surface 32a of the base 32 along the longitudinal axis L. The lateral wall 42a is continuous, but as an alternative embodiment it could be discontinuous. A height of the lateral wall 42a of the protective element 40 in relation to the base 32 is less than a length of the tabs 35 in the deployed position. The tabs 35 in the deployed position therefore protrude from the lateral wall 42a of the protective element 40. However, the lateral wall 42a of the protective element 40 is configured to be flush with the contact parts 35b of the tabs 35 in the retracted position, when the sensor 30 is placed on the anatomical zone.

The locking member 45 of the sensor 30 is distinct from the electrical connection member 38.

In the embodiment shown, it is integrated in the protective element 40. In particular, the locking member 45 comprises two locking rods 46 extending from a portion of the protective element 40 covering the rim of the base 32, along the longitudinal axis L in a direction opposite to the tabs 35.

Each of the locking rods 46 is configured to cooperate with one of the locking edges 27 and the corresponding locking opening 26 on the helmet 11. The locking rod 46 has a locking surface 47 that is transverse in relation to the longitudinal axis L and is facing the base 32. In the figures, the locking surface 47 is arranged on a lug 48 at a free end of the locking rod 46.

In order to fix the sensor 30 onto the helmet 11, the sensor 30 is positioned facing the indentation 16, coaxial therewith, in a first angular position of the locking rods 46 in relation to the locking edges 27 along the longitudinal axis L. In this first angular position, each locking rod 46 can pass through the corresponding locking opening 26. The sensor 30 is then placed in the indentation 16, with the external edge 33 of the base 32 covered by the protective element 40 facing the lateral surface 17 of the indentation 16, the fitting wall 34 of the base 32 in the connection opening 20 and the locking rods 46 in the locking openings 26. The sensor 30 is pivoted along the longitudinal axis L toward a second angular position, in which the locking rods 46 are prevented from passing through the locking openings 26. The locking surfaces 47 of the locking rods 46 are then in abutment on the locking edges 27 in order to fix the sensor 30 onto the helmet 11. Resilient members can be provided in the helmet 11 in order to urge the locking surfaces 47 of the locking rods 46 toward the locking edges 27.

In this position, the contact strips 19 of the helmet 11 come into contact with the electrical connection members 38 of the sensors to ensure the transmission of the measured biological potentials from the tabs 35 to the processing unit 2.

As an alternative embodiment, any other arrangement of one or more locking rods 46 and corresponding locking openings 26 could be provided.

Furthermore, any other locking member distinct from the electrical connection member can be provided on the protective element 40 or directly on the base 32.

For example, the locking member could comprise at least one locking rod extending from the base, directly or by means of the protective element, along the longitudinal axis in a direction opposite to the tab and a detachable pin. The locking rod would then comprise a locking orifice extending transversally in relation to the longitudinal axis. The locking rod and the pin would be configured to cooperate with a locking hole as a complementary locking member on the helmet, with the pin extending into the correspondingly placed locking orifice and locking hole when the sensor is fixed onto the helmet.

According to another example, the locking member could comprise a thread on a lateral wall extending from the base, directly or by means of the protective element, along the longitudinal axis in a direction opposite to the tab. The thread would be adapted to cooperate with a complementary thread as a complementary locking member on the helmet.

According to another example, the locking member could be a clip. The clip would comprise at least one locking rod extending from a surface of the base opposite to the tab, directly or by means of the protective element. The locking rod then would be configured to cooperate with a locking edge as a complementary locking member on the helmet. In particular, the locking rod would have a rest position, in which said locking rod extends along the longitudinal axis and has a locking surface transverse to the longitudinal axis and facing the base. The locking rod would be resiliently deformable in order to be spaced apart from the rest position, with the locking surface being in abutment on the locking edge when the sensor is fixed onto the helmet.

According to another example, the locking member could comprise a crimping skirt extending from a surface of the base opposite to the tab, directly or by means of the protective element. The crimping skirt would be configured to cooperate with a locking edge as a complementary locking component on the helmet. In particular, the crimping skirt would have an assembly state, in which it defines a housing around the longitudinal axis that is adapted to accommodate the locking edge. The crimping skirt would be deformable in order to have at least one locking surface transverse to the longitudinal axis and would be arranged to retain the crimping edge in the housing when the sensor is fixed onto the helmet.

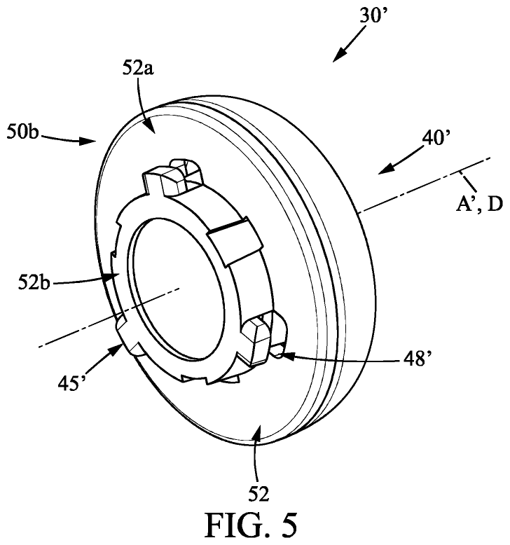

FIGS. 4 and 5 show a sensor 30′ according to a second embodiment of the invention.

In addition to the previously described measurement electrode 31, the sensor 30′ according to the second embodiment comprises a casing 50 receiving a portion of the measurement electrode 31. In particular, the casing 50 comprises a frontal casing portion 51 and a dorsal casing portion 52 configured to be assembled together in a detachable manner whilst defining a housing 53 accommodating the base 32, a portion of the tabs 35 and at least one portion of the electrical connection member 38 of the measurement electrode 31.

The dorsal casing portion 52 has a peripheral portion 52a around a casing axis D aligned with the longitudinal axis L of the sensor 30, and a central portion 52b centered on the casing axis D. The central portion 52b is offset along the casing axis D in relation to the peripheral portion 52a in order to form a portion of the housing 53 on a first face of the dorsal casing portion 52.

On a second face opposite to the first face, the dorsal casing portion 52 supports a dorsal surface 50b of the casing 50, extending both over the peripheral portion 52a and over the central portion 52b. The dorsal surface 50b is intended to be placed facing the external support 10 and comprises the locking member 45′ shaped to form a bayonet attachment as previously described. In the second embodiment, the locking member 45′ is configured to cooperate with one of the locking edges 27 and the corresponding locking opening 26 on the helmet 11. In particular, it comprises an annular locking skirt around the casing axis D and from which three evenly distributed lugs 48′ extend radially, in order to each have a locking surface 47 transverse to the casing axis D.

The frontal casing portion 51 is provided with one or more orifices 54 arranged to allow the passage of the tabs of the measurement electrode 31 when it is placed in the housing 53. The frontal casing portion 51 is shaped to be mounted onto the central portion 52b of the dorsal casing portion 52 so that an external surface 51a, opposite to the dorsal casing portion 52, is flush with a first frontal surface portion of the peripheral portion 52a of the dorsal casing portion 52, opposite to the dorsal surface 50b. The frontal casing portion 52b thus supports a second frontal surface portion, which forms, with the first frontal surface portion of the peripheral portion 52a of the dorsal casing portion 52, a frontal surface 50a of the casing 50, opposite to the dorsal surface 50b.

The measurement electrode is mounted in the housing 53 between the frontal 51 and dorsal 52 casing portions, with its tabs 35 projecting in relation to the frontal surface 50a of the casing 50.

Furthermore, in the second embodiment, the protective element 40′ is secured to the casing 50 so as to extend from the frontal surface 50a of the casing 50, whilst fully covering said surface. In particular, the protective element 40′ comprises a bottom 43′ extending transversally in relation to the protective element axis A′. The bottom 43′ is provided with one or more orifices 44′ arranged to allow the passage of the tabs of the measurement electrode 31. The protective element 40′ also comprises the annular bead 42′ extending from the bottom 43′ around the protective element axis A′. The bead 42′ has the lateral wall 42a′ that defines, with the bottom 43′, the cavity 41′, into which the tabs 35 extend.

The invention has been described in relation to a measurement system that is adapted for measuring a biological potential on the skull of an individual with a view to characterizing sleep. The invention is nevertheless applicable to measuring any other biological potential particularly, but not exclusively, with a view to controlling, characterizing, monitoring and/or feeding back on health parameters, such as cardiac or brain parameters. The external support is then adapted accordingly and can be in any suitable form other than a helmet, and in particular a bracelet or a belt.

1. A sensor for measuring a biological potential of an individual, the sensor being intended to be placed on an anatomical zone of the individual by means of an external support, the sensor comprising:
a measurement electrode adapted to conduct an electric potential, said measurement electrode comprising:
a base;
at least one tab extending from the base to a free contact part intended to come into contact with the anatomical zone of the individual, the tab being configured so that at least the contact part can move relative to the base; and
at least one electrical connection member configured to cooperate with at least one complementary electrical connection member on the external support;
at least one locking member configured to reversibly cooperate with at least one complementary locking member on the external support so as to detachably fix said sensor to the external support, the locking member being separate from the electrical connection member;
wherein said sensor further comprises a protective element extending from the base and defining a cavity, into which the tab extends, the protective element being configured to be flush with the contact part of the tab when the sensor is placed on the anatomical zone, the protective element being produced from a resilient material.

2. The sensor as claimed in claim 1, wherein the protective element comprises the locking member.

3. The sensor as claimed in claim 1, wherein the resilient material has a Young's modulus ranging between 10 Kpa and 100 MPa, in particular between 10 Kpa et 80 MPa, the resilient material particularly being a silicone, a plastic material or an elastomer.

4. The sensor as claimed in claim 1, having a longitudinal axis and wherein the locking member and the complementary locking member are configured to form a bayonet attachment, the locking member comprising at least one locking surface extending transversely in relation to the longitudinal axis and facing the base in a direction opposite to the tab, the locking surface being configured to cooperate with a locking edge as a complementary locking member on the external support, the locking edge defining a locking opening, the locking member and the locking edge being shaped to allow the locking surface to pass through the locking opening in a first angular position of the locking member in relation to the locking edge along the longitudinal axis, and to prevent the locking surface from passing through the locking opening in a second angular position of the locking member in relation to the locking edge along the longitudinal axis, the locking surface being in abutment on the locking edge when the sensor is fixed onto the external support in the second angular position.

5. The sensor as claimed in claim 4, wherein the locking member comprises at least two locking rods extending from the base along the longitudinal axis, each locking rod having the locking surface.

6. The sensor as claimed in claim 4, wherein the locking member comprises an annular locking skirt around the longitudinal axis and from which at least two lugs radially extend so as to each have the locking surface.

7. The sensor as claimed in claim 1, having a longitudinal axis and wherein the locking member comprises at least one locking rod extending from the base along the longitudinal axis in a direction opposite to the tab, and a detachable pin, the locking rod comprising a locking orifice extending transversely in relation to the longitudinal axis, the locking rod and the pin being configured to cooperate with a locking hole as a complementary locking member on the external support, the pin extending into the correspondingly placed locking orifice and locking hole when the sensor is fixed onto the external support.

8. The sensor as claimed in claim 1, having a longitudinal axis and wherein the locking member comprises a thread on a lateral wall extending from the base along the longitudinal axis in a direction opposite to the tab, the thread being adapted to cooperate with a complementary thread as a complementary locking member on the external support.

9. The sensor as claimed in claim 1, having a longitudinal axis and wherein the locking member is a clip, the clip comprising at least one locking rod extending from a surface of the base opposite to the tab, the locking rod being configured to cooperate with a locking edge as a complementary locking member on the external support, the locking rod having a rest position, in which said locking rod extends along the longitudinal axis and has a locking surface transverse to the longitudinal axis and facing the base, the locking rod being resiliently deformable in order to be spaced apart from the rest position, the locking surface being in abutment on the locking edge when the sensor is fixed onto the external support.

10. The sensor as claimed in claim 1, having a longitudinal axis and wherein the locking member comprises a crimping skirt extending from a surface of the base opposite to the tab, the crimping skirt being configured to cooperate with a locking edge as a complementary locking member on the external support, the crimping skirt having an assembly state, in which said crimping skirt defines a housing around the longitudinal axis that is adapted to receive the locking edge, the crimping skirt being deformable so as to have at least one locking surface transverse to the longitudinal axis and being arranged to retain the crimping edge in the housing when the sensor is fixed onto the external support.

11. The sensor as claimed in claim 1, wherein the base has a central axis and the tab comprises an attachment part secured to the base, the contact part being mounted so as to translationally move along the central axis on the attachment part.

12. The sensor as claimed in claim 1, wherein the base has a central axis and the contact part is translationally movable in relation to the central axis.

13. The sensor as claimed in claim 1, wherein the measurement electrode is a dry electrode.

14. The sensor as claimed in claim 1, wherein the measurement electrode is made up of a conductive material or a material that is rendered conductive and is selected from the list of materials formed by at least one metal material or a metal alloy and/or at least one polymer.

15. The sensor as claimed in claim 14, wherein the material of the measurement electrode is rendered conductive by the at least partial application of a conductive layer particularly selected from a paint, an ink, a glue and an adhesive.

16. The sensor as claimed in claim 15, wherein the conductive layer comprises silver, silver salts, silver derivatives or a silver alloy, in particular an ink based on silver chloride.

17. The sensor as claimed in claim 15, wherein the conductive layer comprises a conductive polymer, preferably a mixture of poly(3,4-ethylenedioxythiophene) (PEDOT) and of sodium poly(styrene sulfonate) (PSS).

18. The sensor as claimed in claim 1, wherein the electrical connection member is either a part made of conductive material overmolded by the material forming the measurement electrode or is produced directly from the material forming the measurement electrode.

19. The sensor as claimed in claim 18, wherein the overmolded part forming the electrical connection member is made up of at least one metal material or of at least one metal alloy.

20. The sensor as claimed in claim 1, further comprising a casing accommodating the base, a portion of the tab and at least one portion of the electrical connection member of the measurement electrode, the casing having a dorsal surface intended to be placed facing the external support, and a frontal surface opposite to the dorsal surface, the tab of the measurement electrode projecting in relation to the frontal surface of the casing, the protective element being secured to the casing and extending from the frontal surface of the casing.

21. The sensor as claimed in claim 20, wherein the casing comprises the locking member.

22. The sensor as claimed in claim 21, wherein the locking member extends from the dorsal surface of the casing.

23. The sensor as claimed in claim 20, wherein the casing comprises a frontal casing portion and a dorsal casing portion configured to be detachably assembled together by defining a housing accommodating the base, a portion of the tab and at least one portion of the electrical connection member of the measurement electrode, the frontal casing portion supporting at least one portion of the frontal surface of the casing and the dorsal casing portion supporting at least one portion of the dorsal surface of the casing, the frontal casing portion being provided with at least one orifice, through which the tab of the measurement electrode extends.

24. The sensor as claimed in claim 23, wherein the dorsal casing portion has a peripheral portion around a casing axis and a central portion centered on the casing axis, with the central portion being offset along the casing axis in relation to the peripheral portion in order to form a portion of the housing, the frontal casing portion being mounted on the central portion of the dorsal casing portion.

25. The sensor as claimed in claim 20, wherein the protective element comprises a bottom extending transversely in relation to a protective element axis and a lateral wall extending from the bottom around the protective element axis, the protective element covering the frontal surface of the casing and being provided with at least one orifice arranged in the bottom and through which the tab of the measurement electrode passes.

26. A measurement assembly comprising a sensor as claimed in claim 1 and an external support configured to be placed on an anatomical zone of the individual, the external support comprising:
at least one complementary electrical connection member configured to cooperate with the connection member of the sensor;
at least one complementary locking member configured to reversibly cooperate with the locking member of the sensor.

27. The measurement assembly as claimed in claim 26, wherein the external support is selected from a helmet, a bracelet and a belt.

28. A measurement system comprising a measurement assembly as claimed in claim 26 and a processing unit configured to receive the biological potential measured by the sensor.
